(19) United States
(12) Patent Application Publication
(10) Pub. No.: US 2025/0090264 A1
(43) Pub. Date: Mar. 20, 2025

(54) Damper and Surgical Imaging Device

(71) Applicant: Leica Instruments (Singapore) Pte Ltd., Singapore (SG)

(72) Inventor: Roman FRICK, Singapore (SG)

(21) Appl. No.: 18/889,490

(22) Filed: Sep. 19, 2024

(30) Foreign Application Priority Data
Sep. 19, 2023 (DE) 102023125411.1

(51) Int. Cl. A61B 90/00

(52) U.S. Cl. CPC A61B 90/36; A61B 2217/002

(57) ABSTRACT

The present disclosure relates to a damper for a surgical imaging device and a surgical imaging device. The damper is comprising a stack of layers comprising: a first outer layer, a central layer, and a second outer layer. The layers are linked to form a base piece of the damper. The first outer layer further forms a first end of the damper configured to be connected to a stand of the surgical imaging device and the second outer layer further forms a second end of the damper configured to be connected to a base of the surgical imaging device. The damper is configured to undergo elastic deformation in response to a pushing or pulling force exhibited on one of the ends such that the central layer, but not the outer layers, undergo elastic deformation. Application of the force to the first end causes a deferral of the first outer layer relative to the second outer layer, thereby inducing shearing deformation of the central layer.

### TECHNICAL FIELD

The present disclosure relates to a damper for a surgical imaging device and a surgical imaging device.

### BACKGROUND

In an operating room, surgical imaging devices, such as surgical microscopes, are subjected to various impacts, especially in their use time during surgery. Aside from chemical stress caused by fluid or chemical spills, the surgical imaging device comprises components, such as a fan for cooling of electronic parts or a ventilation system for drape suction, which cause slight vibration of the surgical imaging device. Usually, these vibrations can be overcome by image stabilization.

However, significant mechanical stress may also be exerted by the user while handling the surgical imaging device with or without his intend. For example, the user might accidentally hit parts of the surgical imaging device when moving around during a surgical procedure. These impacts may result in considerable or even heavy shaking of the surgical imaging device. Thereby, the user may be precluded from obtaining optimal imaging data from the operating site until the surgical imaging device returns to a steady state. The user might even have to wait until the surgical imaging device returns to a still before he might use it again for the intended surgical procedure.

Therefore, further efforts are required for dealing with mechanical stress exerted on the surgical imaging device.

### SUMMARY

There may be a desire for an improved concept for damping mechanical impacts, which are exerted on the surgical imaging device.

This desire is addressed by the subject-matter of the independent claims.

Embodiments of the present disclosure provide a damper for a surgical imaging device. The damper comprises a stack of layers comprising: a first outer layer, a central layer, and a second outer layer. The layers are linked to form a base piece of the damper. The first outer layer further forms a first end of the damper configured to be connected to a stand of the surgical imaging device and the second outer layer further forms a second end of the damper configured to be connected to a base of the surgical imaging device. The damper is configured to undergo elastic deformation in response to a pushing or pulling force exhibited on one of the ends such that the central layer, but not the outer layers, undergo elastic deformation. Application of the force to the first end causes a deferral of the first outer layer relative to the second outer layer, thereby inducing shearing deformation of the central layer.

With the damper being configured to be mounted inside the surgical imaging device such that the base and the stand are connected to one of the ends of the damper, a movement of the stand and/or the base is acting on the damper resulting in its shearing deformation. The movement may be caused by a mechanical impact on the surgical imaging device. This may occur for example when a user accidentally hits parts of the surgical imaging device during a medical procedure. Such parts may be an arm or an image acquisition apparatus carrier, like an optics carrier or a camera, of the surgical imaging device. These impacts may then be transmitted through the surgical imaging device as the respective parts, the stand and the base are mechanically linked. By that a movement of the stand and/or the base may be generated, which creates the pushing or pulling force that is acting on at least one of the ends of the damper. As a result, the damper undergoes shearing deformation, thereby changing the shape of the central layer, but not of the outer layers. It is understood that shearing deformation encompasses shearing and deshearing of the damper. In this context, deshearing is a spontaneous return of the damper to its undeformed state after its deformation or shearing. By returning to its undeformed state, not only the deformation of the damper is reversed, but also the movement of the stand and/or base, which are connected to the ends of the damper. Hence, the damper contributes to a return of the surgical imaging device to a steady state following an impact. This improves usability of the surgical imaging device during a medical procedure as the user may be able to operate the surgical imaging device instantly or at least quickly after an impact has occurred.

Embodiments of the present disclosure further provide a surgical imaging device. The surgical imaging device comprises a stand, a base, and the damper.

The surgical imaging device may be a surgical microscope placed in an operating room. The surgical imaging device may further comprise an arm and an image acquisition apparatus carrier, like an optics carrier or a camera. During a surgical procedure medical personnel, such as surgeons or surgery assistants, move around the surgical imaging device and, by chance, may hit one of the aforementioned parts of the surgical imaging device causing a mechanical impact. By that a pushing or pulling for is exerted on the respective part. The force is then transmitted through the surgical imaging device. Eventually, the pushing or pulling force is transmitted to the damper, which is connected to the stand and the base by its ends. Through shearing of the damper in response to the force and subsequent spontaneous deshearing, the damper contributes to a return of the surgical imaging device to a steady state after such impact has occurred.

### SHORT DESCRIPTION OF THE FIGURES

Some examples of apparatuses and/or methods will be described in the following by way of example only, and with reference to the accompanying figures, in which

FIG. 1 shows a schematic perspective view of a damper according to an embodiment;

FIG. 2 shows the damper of FIG. 1 in a schematic side view;

FIG. 3 shows a schematic perspective view of the damper of the preceding figures in a deformed state;

FIG. 4 shows the damper of FIG. 3 in a schematic side view;

FIG. 5 shows a schematic side view of a surgical imaging device according to an embodiment;

FIG. 6 shows a schematic perspective view of the surgical imaging device of FIG. 5 with the damper of the preceding figures;

FIG. 7 shows another schematic perspective view of the damper of the preceding figures mounted within the surgical imaging device of FIG. 5;

FIG. 8 shows the damper inside the surgical imaging device as in FIGS. 6 and 7 in a schematic sectional view;

FIG. 9 shows another schematic sectional view of the damper inside the surgical imaging device as in FIGS. 6 and 7;

FIG. 10 shows a schematic sectional view of the surgical imaging device with the damper of the preceding figures.

### DETAILED DESCRIPTION

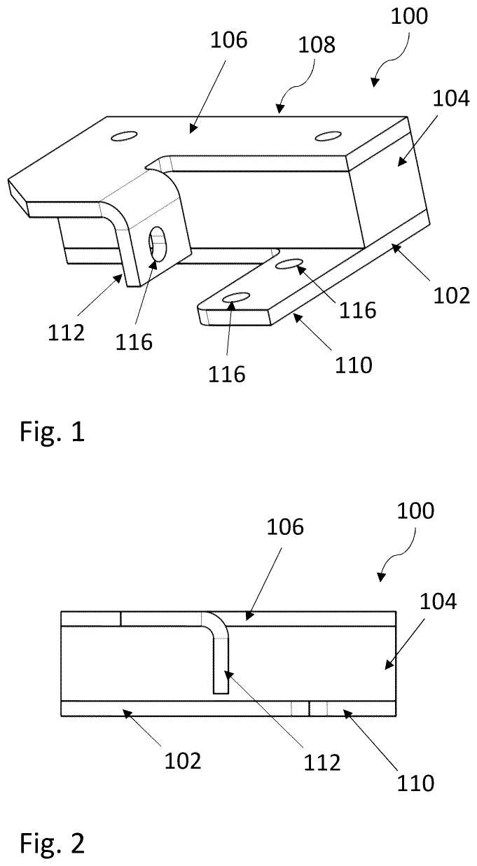

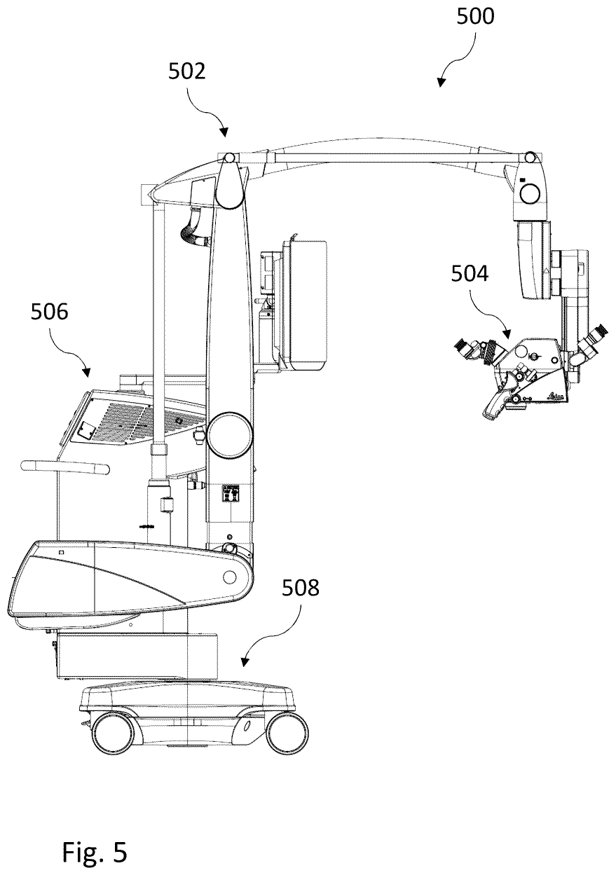

A surgical imaging device 500 (as shown in FIG. 5), such as a surgical microscope, may be used in an operating room during a medical procedure. A user of the surgical imaging device 500, like a surgeon or surgery assistant, may thereby accidentally hit parts of the surgical imaging device 500. These parts of the surgical imaging device 500 may comprise an arm 502 and an image acquisition apparatus carrier 504, like an optics carrier or a camera, a stand 506, and a base 508. Due to the mechanical linking, hitting of one of the aforementioned parts results in a transmission of the applied force through the surgical imaging device 500. To suppress a shaking or movement of the surgical imaging device 500 resulting from the transmitted force, the surgical imaging device 500 may further comprise a damper 100 as further described in the embodiments in FIG. 1ff.

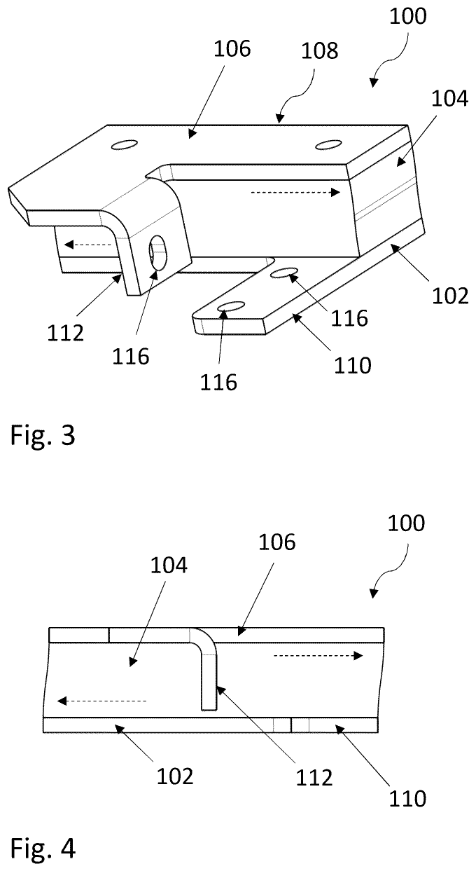

FIG. 1 to FIG. 4 show perspective views of a damper 100 according to an embodiment. FIG. 1 and FIG. 2 show the damper 100 in an undeformed (resting or initial) state and FIG. 3 and FIG. 4 show the damper 100 a deformed state.

The damper 100 comprises a stack of layers 102, 104, 106. The stack comprises a first outer layer 102, a central layer 104, and a second outer layer 106. The layers 102, 104, 106 are linked to form a base piece 108 of the damper 100. Linking of the layers 102, 104, 106 can be realized by a material or form fitting connection, which is known by a person skilled in the art. The first outer layer 102 further forms a first end 110 of the damper 100 configured to be connected to the stand 506 of a surgical imaging device 500 (as shown in FIG. 5ff). The second outer layer 106 further forms a second end 112 of the damper 100 configured to be connected to the base 508 of the surgical imaging device 500 (as shown in FIG. 5ff). By this connection, a pushing or pulling force exerted on the stand 506 and/or the base 508 acts on the damper 100 as it is transmitted via the ends 110, 112. The damper 100 is configured to undergo elastic deformation in response to that pushing or pulling force such that the central layer 104, but not the outer layers 102, 106, undergo elastic deformation.

In a resting state (as shown in FIG. 1 and FIG. 2) the damper 100 and its central layer 104 remain undeformed as no or no substantial force is applied on the ends 110, 112. However, in case of a mechanical impact on the surgical imaging device 500, e.g., from a user hitting parts of the surgical imaging device 500, a movement of the stand 506 and/or the base 508 may occur. Due to the connection between damper 100 and the stand 506 and the base 508 a pushing or pulling force at the ends 110, 112 is created. An application of such force to the first end 110 results in a deferral of the first outer layer 102 relative to the second outer layer 106, thereby inducing shearing deformation of the central layer 104 (as shown in FIG. 3 and FIG. 4, arrows with dashed line indicate the deformation). To remain undeformed, the outer layers 102, 106 may be made of sheet metal. The central layer 104 may be made of an elastomer, preferably polyurethane, to be deformable. The deformation may occur in the direction of the force, so that the central layer 104 may undergo shearing deformation coaxially to a displacement of the first end 110. In further embodiments other materials for the central layer 104 and/or the outer layers 102, 106 may be selected. The outer layers 102, 106 may be configured to undergo no, no significant or at least less deformation than the central layer 104 in response to the force.

The deferral of the first outer layer 102 relative to the second outer layer 106 may thereby be limited to 2 mm. Such deferral of maximum 2 mm may translate to a maximum movement of parts of the surgical imaging device of about 20 mm in the direction of the force. Such parts may be an arm 502 and/or an image acquisition apparatus carrier 504, e.g., a camera, optics carrier, or the like, of the surgical imaging device 500. Hence, in a scenario where the arm and/or image acquisition apparatus carrier 504 of the surgical imaging device 500 are hit (accidentally) by a user and undergo a typical displacement of 2 to 3 mm, the resulting displacement of the damper 100 would range between 0.2 to 0.3 mm. However, it is understood that the deferral of the damper 100 and the related displacement of parts of the surgical imaging device 500 may not be limited to a ratio of 1:10, but other ratios may be included by this disclosure in further embodiments.

Furthermore, the central layer 104 is configured to undergo deshearing following shearing deformation to return from a deformed state, which equals a sheared state of the damper 100 (see FIG. 3 and FIG. 4), to an undeformed state, which equals an unsheared state of the damper 100 (see FIG. 1 and FIG. 2). In an unsheared or undeformed state, the deferral of the outer layers 102, 106 may or may almost be 0 mm. The central layer 104 can repeatedly undergo shearing and deshearing in response to reoccurring pushing or pulling forces applied to the ends 110, 112. Deshearing may occur spontaneously after shearing of the central layer 104 because of the material properties of the central layer 104. Hence, use of an elastic material, such as polyurethane or materials of similar properties, may be preferred for the central layer 104. By returning to its desheared, undeformed state, the damper 100 may act on the stand 506 and/or the base 508 of the surgical imaging device 500 such that the surgical imaging device 500 may return to a steady state after the damper-deforming impact has occurred. This return to the steady state of the surgical imaging device 500 may only take one to a few seconds after the impact and with few or no extra cycles of back-and-forth movement of the respective parts 502, 504, the stand 506 and/or the base 508 of the surgical imaging device 500. Thereby, the surgical imaging device 500 becomes usable again instantly or quickly after an impact has occurred.

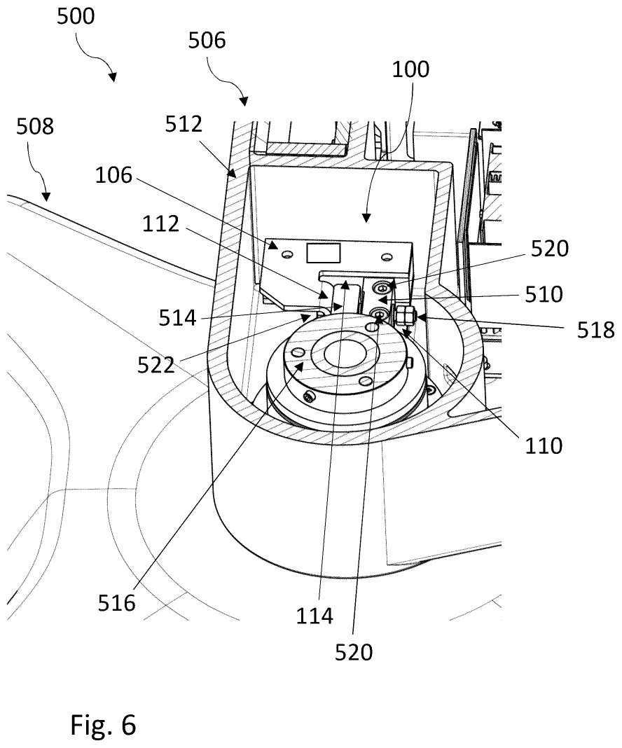

FIG. 6 shows a perspective view of a damper 100 mounted within a surgical imaging device 500. The surgical imaging device 500 may comprise a fixation element 510 for fixing the first end 110 of the damper 100 to a housing 512 of a stand 506 of the surgical imaging device 500. The surgical imaging device 500 may further comprise a further fixation element 514 for fixing the second end 112 of the damper 100 to a connector part 516 of a base 508 of the surgical imaging device 500, with the connector part 516 configured to connect the base 508 with the stand 506. And the surgical imaging device 500 may comprise an axis 518 configured to movably connect the first end 110 and the second end 112. Thereby, both ends 110, 112 are extending from the same side 114 of the base piece 108 of the damper 100. The second end 112 is configured to be movably connected to the first end 110. The movement of the ends 110, 112 may occur along the axis 518 in the direction of the force. The fixation elements 510, 514 may be attached to the ends 110, 112 by mechanical means, such as screws 520 or a bolted connection 522, which may be gearing into respective openings 116 of the ends 110, 112 (see also FIG. 1 and FIG. 3). By that the damper 100 may be mounted easily inside the surgical imaging device 500.

The surgical imaging device 500 may further comprise an image acquisition apparatus carrier 504 (see FIG. 5), such as a camera and/or an optics carrier, and an arm 502 (see FIG. 5). The damper 100 is thereby configured to undergo elastic deformation coaxially to a force exerted on the image acquisition apparatus carrier 504 and/or the arm 502. The force may be caused by a mechanical impact, such as a user (accidentally) hitting the image acquisition apparatus carrier 504 and/or the arm 502. The force is then transmitted through the surgical imaging device 500 as the image acquisition apparatus carrier 504, the arm 502, the stand 506, and the base 508 are mechanically linked. Therefore, an impact on the image acquisition apparatus carrier 504 and/or the arm 502 is eventually transmitted to the damper 100, which is connected to the stand 506 and the base 508 with its ends 110, 112.

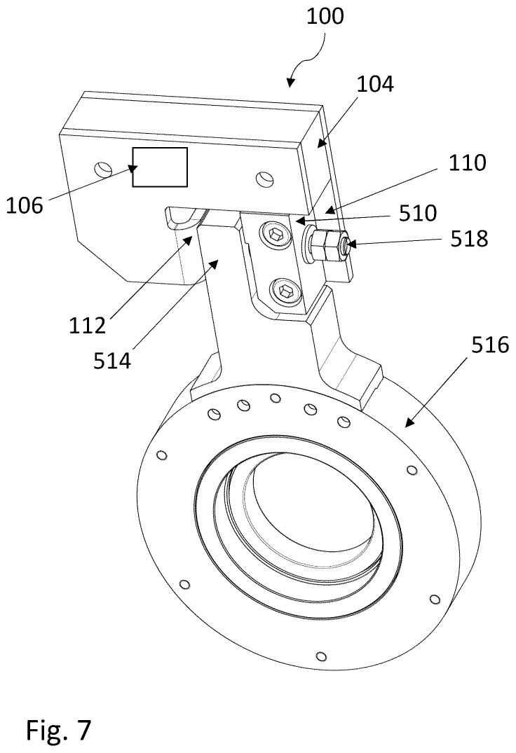

FIG. 7 shows another perspective view of the damper 100 in the context of the surgical imaging device 500. The fixation element 510 is positioned at the first end 110 and the further fixation element 514 at the second end 112 with the axis 518 connecting both ends 110, 112. This is allowing for movement of the ends 110, 112 along the axis 518 in the direction of the force.

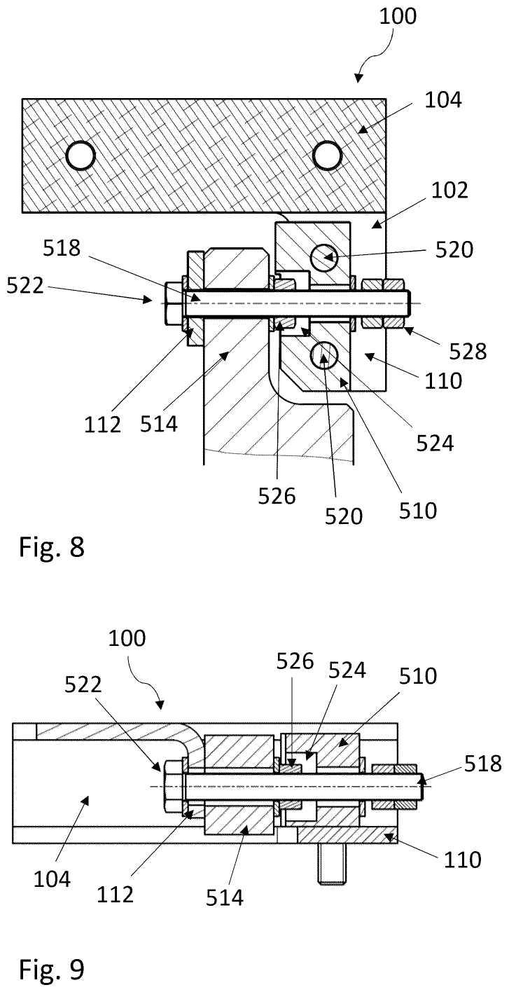

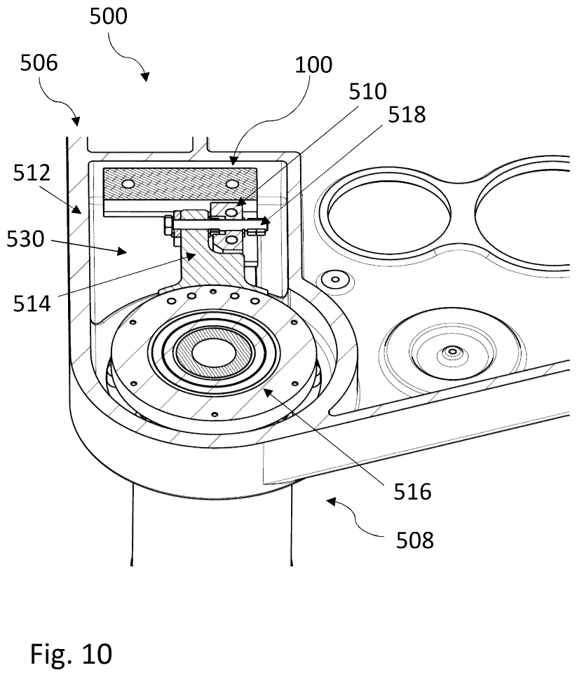

FIG. 8 and FIG. 9 show schematic sectional views of a damper 100 with FIG. 8 showing a top section view and FIG. 9 showing a side section view. The section plane is located at the center of the axis 518 (indicated by dashed lines in FIG. 8 and FIG. 9). In the depicted embodiment, the first end 110 is configured to be fixed at the stand 506 by the fixation element 510. The second end 112 is configured to be fixed at the base 508 by the further fixation element 514. The second end 112 is further configured to be mounted on the axis 518, with the axis 518 being configured to engage with the fixation element 510 in such way that during deformation the first end 110 is displaced along the axis 518 thereby deferring the first outer layer 102 in direction of the force. The fixation elements 510 is attached to the end 110 by two screws 520. The further fixation element 514 is attached to the second end 112 by a bolted connection 522 along with the axis 518. In further embodiments the same or different fixation means may be utilized to attach the fixation elements 510, 514 to the ends 110, 112, which are known to a person skilled in the art. The fixation element 510 may comprise a cavity 524. The cavity 524 may allow for an at least partial take up of the further fixation element 514 and/or parts 526 of the bolted connection 522 when the first end 110 is displaced along the axis 518 towards the second end 112. By this a compact arrangement of the damper 100 inside the surgical imaging device 500 is achieved. The axis 518 may further comprise a mechanical stop 528. The mechanical stop 528 is positioned opposite of the second end 112 and the fixation elements 510, 514. The mechanical stop 528 thereby limits the displacement of the first end 110 along the axis 518. In the depicted embodiment the displacement may be limited such that the deferral of the outer layers 102, 106 is limited to a maximum of 2 mm. Other limits of displacement and therefore other positions of the mechanical stop 528 on the axis 518 may be used in further embodiments. The use of the mechanical stop 528 may prevent over-shearing of the central layer 104 and by that damaging of the damper 100. FIG. 10 shows a sectional view of a surgical imaging device 500. The section plane is at the center of the axis 518 (similar to FIG. 8). In the embodiment, the fixation element 510 is fixing the first end 110 of the damper 100 to the housing 512 of a stand 506. The housing 512 thereby comprises a compartment 530 for taking up the damper 100. The further fixation element 514 is fixing the second end 112 of the damper 100 to the connector part 516 of the base 508. The connector part 516 is configured to connect the base 508 with the stand 506. A movement of the stand 506 and/or the base 508 exerts a pushing or pulling force on at least one of the ends 110, 112 of the damper 100, hence, leading to its deformation.

As used herein the term “and/or” includes any and all combinations of one or more of the associated listed items and may be abbreviated as “/”.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

### LIST OF REFERENCE SIGNS

100 Damper
102 Outer layer
104 Central layer
106 Outer layer
108 Base piece
110 First end
112 Second end
114 Side
116 Opening
500 Surgical imaging device
502 Arm
504 Image acquisition apparatus carrier
506 Stand
508 Base
510 Fixation element
512 Housing
514 Fixation element
516 Connector part
518 Axis
520 Screw
522 Bolted connection
524 Cavity
526 Part
528 Mechanical stop
530 Compartment

1. Damper for a surgical imaging device, the damper comprising a stack of layers comprising: a first outer layer, a central layer, and a second outer layer; wherein the layers are linked to form a base piece of the damper; wherein the first outer layer further forms a first end of the damper configured to be connected to a stand of the surgical imaging device and the second outer layer further forms a second end of the damper configured to be connected to a base of the surgical imaging device; wherein the damper is configured to undergo elastic deformation in response to a pushing or pulling force exhibited on one of the ends such that the central layer, but not the outer layers, undergo elastic deformation; wherein application of the force to the first end causes a deferral of the first outer layer relative to the second outer layer, thereby inducing shearing deformation of the central layer.

2. Damper according to claim 1, wherein the deferral is limited to 2 mm.

3. Damper according to claim 1, wherein the ends are extending from the same side of the base piece of the damper, wherein the second end is configured to be movably connected to the first end.

4. Damper according to claim 3, wherein the first end is configured to be fixed at the stand by a fixation element; wherein the second end is configured to be fixed at the base by at least one further fixation element; wherein the second end is further configured to be mounted on an axis, with the axis being configured to engage with the fixation element in such way that during deformation the first end is displaced along the axis thereby deferring the first outer layer in direction of the force.

5. Damper according to claim 3, wherein the central layer undergoes shearing deformation coaxially to a displacement of the first end.

6. Damper according to claim 1, wherein the outer layers are made of sheet metal, wherein the central layer is made of an elastomer, preferably polyurethane.

7. Damper according to claim 1, the central layer is configured to undergo deshearing following shearing deformation to return from a deformed state to an undeformed state.

8. Surgical imaging device comprising a stand, a base, and a damper according to claim 1.

9. Surgical imaging device according to claim 8, the surgical imaging device further comprising a fixation element for fixing a first end of the damper to a housing of the stand, a further fixation element for fixing a second end of the damper to a connector part of the base, with the connector part configured to connect the base with the stand, and an axis configured to movably connect the first end and the second end.

10. Surgical imaging device according to claim 8, the surgical imaging device further comprising an image acquisition apparatus carrier and an arm, wherein the damper is configured to undergo elastic deformation coaxially to a force exerted on the image acquisition apparatus carrier and/or the arm.
